(19) United States
(12) Patent Application Publication
(10) Pub. No.: US 2026/0117311 A1
(43) Pub. Date: Apr. 30, 2026

(54) TISSUE-BASED PROGNOSTIC ASSAY USED TO IDENTIFY STAGE II MELANOMA PATIENTS AT HIGH OR LOW RISK FOR DISEASE RECURRENCE

(71) Applicant: New York University, New York, NY (US)

(72) Inventors: David Polsky, Ardsley, NY (US); Eva Hernando, New York, NY (US); Yongzhao Shao, Forest Hills, NY (US); Jennifer Wiggins, Pleasantville, NY (US)

(21) Appl. No.: 19/373,294

(22) Filed: Oct. 29, 2025

Related U.S. Application Data
(60) Provisional application No. 63/713,350, filed on Oct. 29, 2024.

(51) Int. Cl. C12Q 1/6886

(52) U.S. Cl. CPC C12Q 1/6886; C12Q 2600/118; C12Q 2600/158; C12Q 2600/178

(57) ABSTRACT

Provided are approaches to determine if an individual who has a Stage II cutaneous melanoma tumor is at risk for relapse of the melanoma. The determination is made using a recurrence-prediction model using clinical characteristics in combination with microRNA levels. Treating individuals who are at risk of recurrence with immune checkpoint inhibition is included.

### CROSS-REFERENCE TO RELATED APPLICATIONS

This application claims the benefit of U.S. provisional patent application No. 63/713,350, filed Oct. 29, 2024, the entire disclosure of which is incorporated herein by reference.

### STATEMENT REGARDING FEDERALLY SPONSORED RESEARCH OR DEVELOPMENT

This invention was made with government support under P50 CA225450, P30 CA016087 and T32 AR064184 awarded by the National Institutes of Health. The government has certain rights in the invention.

### RELATED INFORMATION

In December 2021, adjuvant pembrolizumab (an anti PD-1 immunotherapy) was approved for stages IIB and IIC melanoma. The approval was based on the KEYNOTE-716 randomized clinical trial (n=976), which demonstrated an improvement in 18-month recurrence-free survival for pembrolizumab (85.8%) compared with placebo (77.0%). Close examination of these findings reveals that the number needed-to-treat (NNT) to prevent one recurrence was 11:1. The main factor driving this ratio is not the lack of effectiveness of the treatment, but the relatively low recurrence rate in the placebo group (23%). Given the treatment-related toxicity risks (16% grade 3-4 events that led to treatment discontinuation in the trial) and the very high costs of immunotherapy, we need to more accurately identify stage II melanoma patients who may benefit from adjuvant therapy, and distinguish them from surgically cured patients who do not need it. Unfortunately, the commercial molecular tests that have been developed to guide the management of localized melanoma are unproven, as evidenced by the lack of recommendation by the National Comprehensive Cancer Network Melanoma Guidelines, and the American Academy of Dermatology Melanoma Guidelines. Taken together, these factors show the importance of developing new prognostic biomarkers for stage II melanoma, and methods of selecting and treating patients who are likely to benefit from immune checkpoint inhibition. The present disclosure is pertinent to this need.

### BRIEF SUMMARY

This disclosure provides methods for determining whether or not an individual who has Stage II cutaneous melanoma tumor is at risk for relapse of the melanoma. Individuals who are determined to be at risk for relapse can be treated with, for example, one or more immune checkpoint inhibitors. Individuals who are determined to be a low risk for relapse can be spared the potential toxicities and costs of unnecessary treatment.

### BRIEF DESCRIPTION OF THE FIGURES

The patent or application file contains at least one drawing executed in color. Copies of th is patent or patent application publication with color drawing(s) will be provided by the Office upon request and payment of the necessary fee.

FIG. 1. Graphs showing Stage II melanoma Receiver Operating Characteristics curve from Cox models for the predicted survival of patients at 24- and 36-months post diagnosis with the training set (left) and testing set (right).

FIG. 2. Probability of stage II patient recurrence at 3 years post diagnosis using the test set. Patients were stratified using a cut off for low (blue line) and high (gold line) risk of recurrence using a minimum sensitivity of 0.9 using a) miRNA only or b) miRNA+clinical factors in the test set.

FIG. 3: Waterfall plots from Cox model of the 3-year recurrence prediction in the clinical, miRNA only and miRNA+clinical factors models using Min ValueSe at 0.90 as a low/high risk cutoff.

FIG. 4: Area under the curve of the Receiver Operating Characteristics curve for 48 and 60 months. The graphs are for the training set (left) and the testing set (right).

FIG. 5: Probability of stage II patient recurrence at 3 years post diagnosis using the training set. Patients were stratified using a cut off for low (blue line) and high (gold line) risk of recurrence using a minimum sensitivity of 0.9 using a) miRNA only or b) miRNA+clinical factors in the training set.

FIG. 6: Graphs showing Area under the curve of the Receiver Operating Characteristics curve for 48 and 60 months for the training set (left) and the testing set (right) Data Distribution of the 25 miRNAs in the four control samples across batches and the effect of pre-processing. For each repeated sample from top to bottom, counts are displayed in log scale for raw data, after positive correction, after normalization and the Pearson correlation of the counts of each sample between batches after normalization, *** p<0.001. Note that not all control samples were run on every batch.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

All nucleotide sequences that are referred to herein by way of a database are incorporated herein by reference as they exist in the database as of the filing date of this application. The nucleotide sequences of each microRNA described herein are known in the art. Reference to any microRNA here includes alternate nomenclature used to describe the same microRNA, such alternate nomenclature being apparent to those skilled in the art. The disclosure includes use of any combination of miRNAs that comprise or consist of the described miRNAs.

The term “therapeutically effective amount” as used herein refers to an amount of a described agent to achieve, in a single or multiple doses, the intended purpose of treatment. The amount desired or required will vary depending its mode of administration, patient specifics and the like. Appropriate effective amounts can be determined by one of ordinary skill in the art informed by the instant disclosure using routine experimentation. In embodiments, a therapeutically effective amount of a described checkpoint inhibitor is administered to an individual in need thereof, which may be based at least in part on a determination of risk for cancer recurrence using a described method. As described further below, upon a determination of a likelihood that an individual's Stage II cutaneous melanoma is likely to relapse, the disclosure includes administering a therapeutically effective amount of an immune checkpoint inhibitor to the individual.

In an aspect the disclosure provides a method comprising testing a sample of a patient's Stage II cutaneous melanoma tumor to determine if the patient is at risk for relapse of the melanoma by determining a change in microRNAs (also referred to herein as “miRNAs”) in the sample relative to a value for the same microRNAs obtained from individuals who did not experience relapse of the Stage II cutaneous melanoma. The miRNAs may be any combination of miRNAs disclosed in this description and on the accompanying figures. In embodiments, the described miRNAs are used in conjunction with clinical factors, as further described herein. In a non-limiting embodiment a first combination of microRNAs are tested according to the disclosure and are selected from the group consisting of hsa.miR.93.5p, hsa.miR.575, hsa.miR.29c.3p, hsa.let.7g.5p, hsa.miR.200b.3p, hsa.miR.4521, and hsa.miR.585.3p, and any combination thereof. These miRNAs may be used to make a prediction independent of clinical factors.

In a non-limiting example, a second combination of microRNAs that are tested according to the disclosure and are selected from the group consisting of hsa.miR.1234.3p, hsa.miR.29c.3p, hsa.miR.585.3p, hsa.miR.93.5p, hsa.miR.4521, hsa.let.7g.5p, hsa.miR.1180.3p, hsa.miR.575, hsa.let. 7a.5p, and hsa.miR.27a.3p, and any combination thereof. This combination may be used in conjunction with any clinical factor described herein, including but not necessarily limited to tumor Breslow thickness and ulceration, and all statistical interpretations of the data as described herein, including but not limited to all equations described herein, including but not limited to equations used to determine risk of recurrence.

In an embodiment the disclosure comprises determining a change in the microRNAs and a change in the clinical factors relative to a value for the microRNAs and the clinical factors obtained from individuals who did not experience relapse of the Stage II cutaneous melanoma to determine that the individual is at risk of melanoma relapse. In an alternative embodiment, determining microRNAs from a sample from individual that have the same expression profile as an individual who did experience a relapse may be used to determine that the individual from whom the sample was taken may also be at risk of a relapse. In embodiments a described approach may be used to distinguish individuals who have a high risk, or a low risk, of melanoma recurrence. In examples, the disclosure thus provides for comparing patient sample parameters to a control value. In embodiments, the control may be a standardized curve(s), a cutoff or threshold value, and the like, examples of which are provided herein.

A combination of miRNA expression levels and clinical factors can be used to determine a risk score. In an embodiment the risk score is determined using the following equation:

−0.389*hsa.miR.585.3p−1.68*hsa.miR.4521+0.555*hsa.miR.575+0.573*log(thickness)+0.764*ulceration

Thus, in an embodiment, a change in a risk score relative to a risk score value for an individual or population of individuals who did not experience relapse can be used to select a patient for immune checkpoint therapy. Accordingly, in one embodiment, the expression level of miRNAs hsa.mir.585.3p, hsa.mir.4521, and hsa.mir.575 can be used with tumor thickness and ulceration values to generate a relapse risk score. If the individual is determined to have a risk of melanoma relapse, the disclosure includes administering an immune checkpoint inhibitor to the individual. The immune checkpoint inhibitor includes but is not necessarily limited to an anti-PD-1 immunotherapy. Non-limiting examples of agents used for anti-PD-1 immunotherapy include Nivolumab and Pembrolizumab.

In some examples, the immune checkpoint inhibitor that is used to treat a patient who has been determined to have a described risk may be an anti-PD-1 antibody, an anti-PD-L1 antibody, an anti-CTLA4 antibody, an anti-LAG-3 antibody, an anti-TIM-3 antibody, an anti-VISTA antibody, an anti-B7-H3 antibody, an anti-TIGIT antibody, or any combination thereof. In examples, the anti-PD-1 antibody may be pembrolizumab, nivolumab, cemiplimab, dostarlimab, retifanlimab, or toripalimab. In some examples, the anti-PD-L1 antibody may be avelumab, atezolizumab, or durvalumab. In some examples, the anti-CTLA-4 antibody may be ipilimumab or tremelimumab. In an example, the anti-LAG-3 antibody may be relatlimab. In an example, the anti-TIM-3 antibody is INCAGN02390. In an example, the anti-VISTA KVA antibody is KVA12123. In an example, the anti-B7-H3 antibody is Enoblituzumab. In examples, the anti-TIGIT antibody is Vibostolimab, Etigilimab, or Tiragolumab. Combination of the antibodies may also be administered.

As discussed above, the disclosure includes measuring the described microRNAs. The term “microRNA” can be used interchangeably with “miR,” or “miRNA” and “hsa.mir.” to refer to, for example, an unprocessed or processed RNA transcript from a naturally occurring miRNA gene. The unprocessed miRNA gene transcript is also called a “miRNA precursor,” and typically comprises an RNA transcript of about 70-100 nucleotides in length. The miRNA precursor can be processed by digestion with an RNAse (for example, Dicer, Argonaut, or RNAse III) into an active 19-25 nucleotide RNA molecule. This active 19-25 nucleotide RNA molecule is also called the “processed” miRNA gene transcript or “mature” miRNA. Any of these forms of microRNA can be adapted for use in embodiments of this disclosure. The sequences of all of the microRNAs described herein and their pre- and processed forms are known in the art and can be readily obtained from databases.

The sample from the individual who has stage II melanoma may be any suitable biological sample. In one embodiment, the sample is a tumor sample. The tumor sample may be processed to facilitate identification of the described miRNAs. In a non-limiting approach, formalin-fixed, paraffin-embedded primary melanoma tumors from patients with AJCC stage II melanoma are processed. The process may include microscopic review of a hematoxylin and eosin-stained slide to identify the tumor and perform macrodissection procedure in which surrounding normal tissue is removed from the tumor cells. Approximately 10 unstained sections of tumor material undergo macrodissection, and the remaining tumor material is put into a container. The tumor material undergoes processing to extract nucleic acids. In one embodiment, RNA concentrations are determined using least of 90 ng RNA, which are analyzed using hybridization to capture and reporter probes using a suitable system, such as the Nanostring nCounter® Human v3 miRNA Expression Assay.

The following description Example illustrates embodiments of the disclosure and is not intended to be limiting.

### Example

Patient clinical characteristics. We obtained formalin fixed paraffin embedded melanoma tumors from 198 stage II patients enrolled in the NYU Melanoma Clinicopathological-Biospecimen Database and Repository. Two patients were excluded due to the presence of multiple primary tumors. miRNA expression profiles were assessed on 196 patients using the NanoString platform. One sample failed NanoString analysis and 20 were excluded due to low miRNA expression profiles and were not usable for further analysis. In total 175 tumors from 97 (55.4%) male and 78 (44.6%) female patients were included in the study. The overall median age at diagnosis was 70 years [IQR: 57.5, 77.0], 65 years [IQR: 57.5, 77.0] for those who recurred and 71 years [IQR: 59.5, 78.0] for those that did not recur (Table 1).

Clinical characteristics and associations with recurrence within 3 years from diagnosis. We examined several clinical pathologic variables and their associations with melanoma recurrence within three years of diagnosis. Using a univariate analysis, we found no association between age at diagnosis and recurrence. Similarly, there was no association with sex, or race; although only six patients were classified as non-white. The tumor characteristics, ulceration and tumor infiltrating lymphocytes (TILs) were also not associated with recurrence. However, our analysis showed that the mitotic index was leaning towards significance (p=0.066), with a median index of 6.0 [IQR: 3.0, 8.8] in the recurred group and 4.0 [IQR: 2.0, 8.0] in the non-recurred group. Additionally, two clinical characteristics were statistically significantly associated with recurrence; higher sub-staging (p=0.011) and Breslow tumor thickness (p<0.003) with a median size of 3.5 mm [IQR: 2.3, 4.3] in the recurred group and 2.5 mm [IQR: 2.0, 3.8] in the non-recurred group (Table 1). Recurrence within 3 years of diagnosis was also statistically significantly associated with patients who died of all causes (p<0.001).

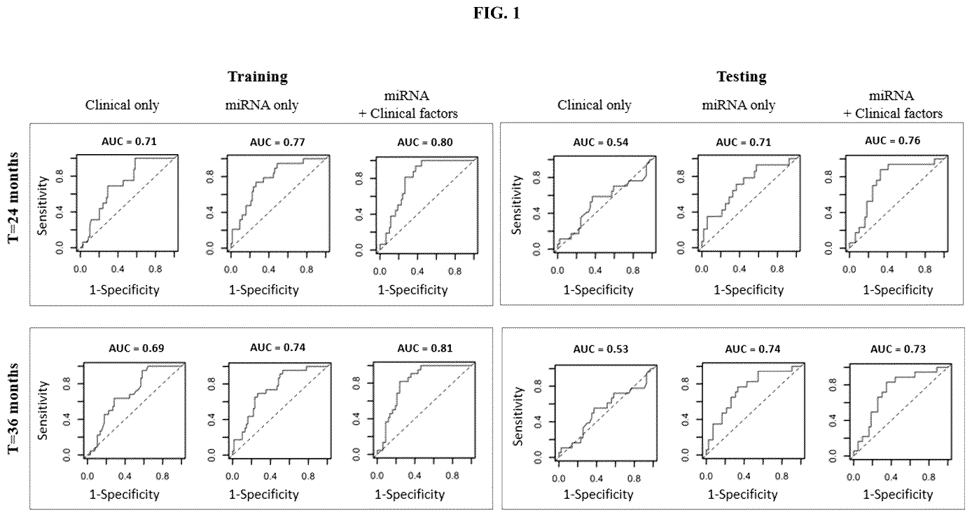

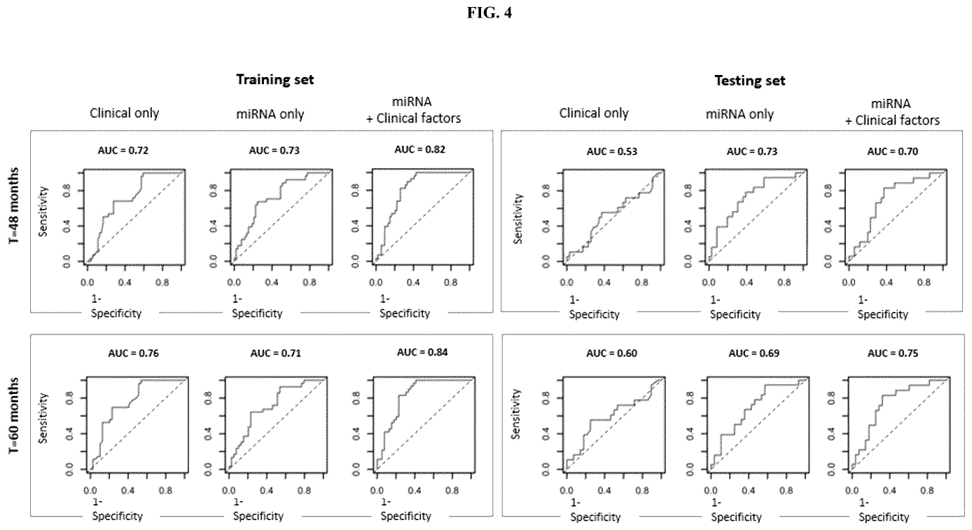

Development of clinical characteristics recurrence prediction model. In order to develop a recurrence-prediction model using clinical characteristics only (hereafter referred to as the clinical only model) the cohort was split into training and testing sets. The training set was used to create the clinical characteristics model and the test set was used to assess the performance of the model. Clinical characteristic, Breslow thickness (log transformed) and ulceration were used to create a time to recurrence prediction model via Cox modeling. For each time point we obtained Receiver Operating Characteristics (ROC) curves and calculated their respective Area Under the Curve (AUC). In the test set the AUCs at 24, 36, 48 and 60 months were 0.54, 0.53, 0.53, 0.60, respectively (FIGS. 1 and 4). Therefore, thickness and ulceration poorly predicted time to recurrence in stage II melanoma patients.

miRNAs associated with Breslow thickness (48 miRNA) and ulceration (17 miRNA). Tumor phenotypic characteristics such as thickness and ulceration may be linked to specific miRNA species. All miRNA counts for each miRNA species were evaluated as described in Data pre-processing. miRNA that were below threshold were excluded from further analysis, which resulted in 195 miRNA species above threshold. To investigate biological associations between miRNAs and tumor characteristics we evaluated each of the 195 miRNAs with log thickness or ulceration using a linear regression analysis. We observed 48 miRNAs associated with log tumor thickness and 17 miRNAs associated with tumor ulceration (data not shown). Next, we sought to determine if miRNA could also be associated with recurrence.

Development of a miRNA only recurrence prediction model. All miRNAs above threshold (n=195) were evaluated using univariate Cox regression analysis. Candidate miRNAs were selected if: i) their coefficients were in the same direction in all sample batches and ii) their p-value was less than 0.1 in at least one of the batches. As a result, we obtained a list of 20 candidate miRNAs (Table S1), which were used to build the recurrence-prediction model. In order to create the miRNA model, the cohort was split into training and a set-aside, validation test set. The training set was used to create 20 sub-training subsets to select miRNA signatures, and the 20 sub-testing subsets were used to assess the predictive accuracy of the preliminary models built from the training subsets. We used Cox modeling with LASSO variable selection to create the preliminary models For each sub-training set, we obtained one time to recurrence prediction model. At each time point and for each model we derived Receiver Operating Characteristics (ROC) curves and calculated their respective Area Under the Curve (AUC). The model with the highest-ranking AUC was selected as the final model.

The miRNA only prediction model with the highest rank includes five miRNAs (hsa-miR-200b-3p, hsa-miR-4521, hsa-miR-575, hsa-miR-93-5p, hsa-miR-29c-3p). Based on their coefficients, two miRNAs were found to be negatively associated with recurrence (hsa-miR-4521 and hsa-miR-29c-3p), and the remaining three were found to be positively associated with recurrence (hsa-miR-200b-3p, hsa-miR-575, hsa-miR-93-5p) (Table 2). In the validation test set, the miRNA only prediction model had an AUC of 0.71 at 24 months; 0.74 at 36 months; 0.73 at 48 months and 0.69 at 60 months (FIGS. 1 and 4). Of note, hsa-miR-200b-3p, hsa-miR-4521 and hsa-miR-93-5p were among the 48 miRNAs associated with tumor thickness.

Development of recurrence prediction model combining miRNA and clinical factors. Due to the stability of the clinical factors, making them important in the staging system, we created and tested a composite model composed of miRNA and clinical factors, and then compared it to the separate clinical only and miRNA only models. To derive this new set of miRNA we analyzed the miRNAs above threshold using univariate Cox regression with or without clinical covariates. Again, candidate miRNAs were selected if: i) their coefficients were in the same direction in all sample batches and ii) their p-value was less than 0.1 in at least one of the batches. This narrowed down the list to 25 candidate miRNAs for their association with melanoma time to recurrence (Table S1). As described above, the recurrence prediction model was built by splitting the training set into sub-training and sub-testing sets and selecting the model with highest ranking AUC. For the miRNA derived with clinical covariates, the model includes three miRNA (hsa-miR-585-3p, hsa-miR-4521, hsa-miR-575) and the clinical variables, Breslow thickness and ulceration (Table 2). Two miRNAs were negatively associated with recurrence (hsa-miR-585-3p, hsa-miR-4521). One miRNA (hsa-miR-575) and the clinical factors, Breslow thickness and ulceration, were positively associated with recurrence.

All shared miRNAs between both models the miRNA only and the miRNA+clinical covariates model had coefficients in the same direction, although the values were slightly different based on the presence or absence of clinical factors.

Similar to the previous model, the miRNAs hsa-miR-585-3p, hsa-miR-4521 were among the 48 miRNAs significantly associated with tumor thickness. No miRNA in the signature was independently associated with ulceration.

Comparison of miRNA and clinical parameter recurrence-prediction model with current clinical model alone. We compared the performance of the described newly derived recurrence prediction models (miRNA or miRNA+clinical factors) to the clinical parameters alone. Using AUC as the initial performance measure we observed that the miRNA models were consistently better at predicting time to recurrence than thickness and ulceration alone (FIGS. 1 and 4). Specifically, the miRNA only model showed an improvement in AUC at 24-months from 0.54 (clinical only) to 0.71 (miRNA only) in the test set. The miRNA+clinical factors model however, outperformed both models with an AUC of 0.76 in the testing set at 24-months. This was an improvement of 0.22 compared to the clinical model alone and 0.05 compared to the miRNA only model. At the 36-month time point, the miRNA only and miRNA+clinical factors models resulted in similar AUCs, of 0.74 and 0.73, respectively. Overall, the combined model (miRNA+clinical factors) had an AUC that was equal or better to the miRNA only model in three out of the four time points assessed (24, 36, 48 and 60 months) (FIGS. 1 and 4).

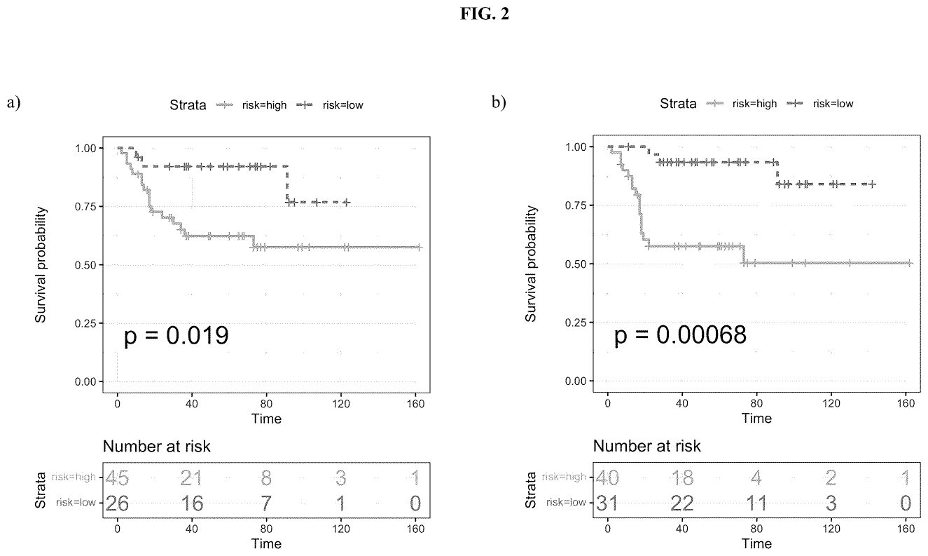

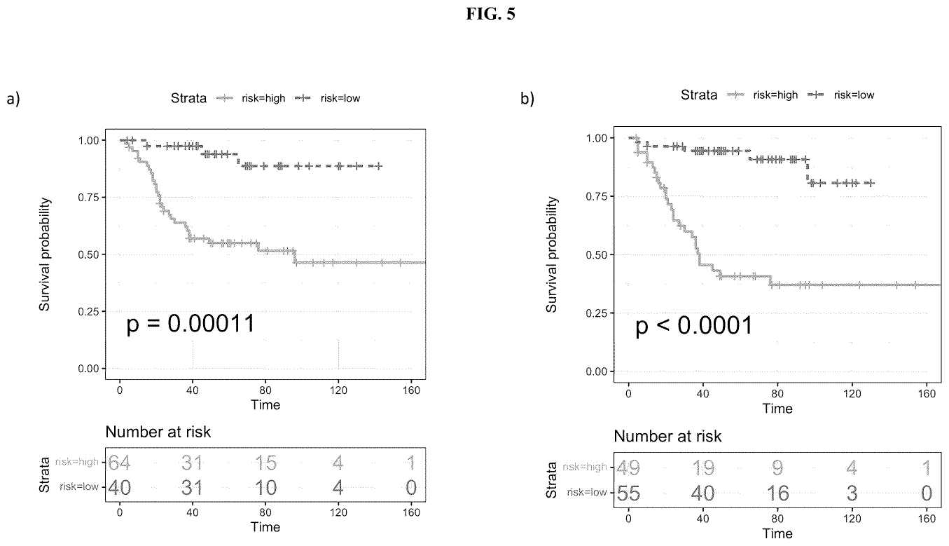

Furthermore, we evaluated the performance of the models using a clinically relevant minimum sensitivity of 90% for clinical decision making. In particular, we chose cutoff values for the calculated risk scores for each patient based on the position on the AUC curve resulting in 90% sensitivity to detect recurrence at 36 months. This means the test would be predicted to correctly classify 90% of recurrent patients as high risk for recurrence. Using this approach, we generated Kaplan-Meier plots which show that miRNAs we were able to stratify patients by high and low risk of recurrence (FIGS. 2 and 5). In particular the miRNAs+clinical factors model was better than the miRNA model at stratifying patients with a p-value of 0.00068 compared to 0.006, respectively. Hence, we moved forward with the combined model for further analysis.

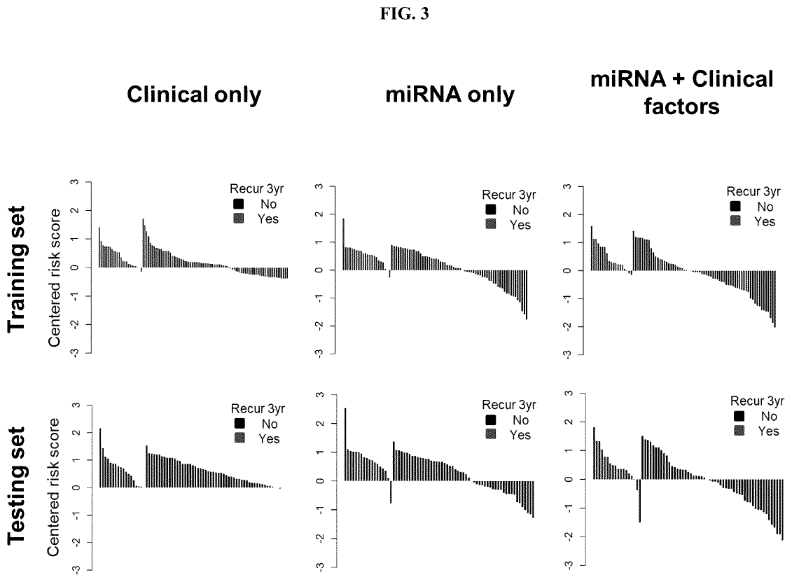

Using the minimum sensitivity to detect recurrence of 90% we plotted the clinical factors model and the combined miRNA+clinical factors model into Waterfall plots to review specificity as well as positive and negative predictive values. Based on the test set, we observed an improvement in specificity from 5% (clinical factors) to 56% (miRNA+clinical factors) (FIG. 3 and Table S2). This improvement in specificity would be predicted to allow a certain percentage of patients to be safely spared adjuvant immunotherapy. In other words, using the clinically relevant high sensitivity of 90%, the accuracy of the model (miRNA & clinical factors) was 63% (45/71) patients in the test set (true positives n=16 and true negatives n=29 out of 71 patients in the test set) vs 28% (20/71) patients in the clinical only model (true positives n=17 and true negatives n=3).

### Discussion of Example

In this disclosure we analyzed a prospective cohort of 175 stage II primary melanoma patients with a minimum follow up of 36 months among non-recurred patients for their miRNA expression patterns. An aspect of the disclosure provides a prediction model combining a miRNA expression signature with clinicopathologic characteristics to predict recurrence among patients with stage II melanoma. Patients were drawn from a well-established institutionally-based prospective cohort with curated patient and tumor information as well as extensive follow up data. The recurrence-prediction model included four miRNAs for the miRNA only analysis (hsa-miR-200b-3p, hsa-miR-4521, hsa-miR-575, hsa-miR-93-5p) and three miRNA for the miRNA+clinical factors analysis (hsa-miR-585-3p, hsa-miR-4521, hsa-miR-575) and the clinical staging factors: Breslow thickness and ulceration.

We used a clinically meaningful sensitivity to detect recurrence of 90% or more to identify patients that will recur within 2 to 5 years of diagnosis and found that the miRNA models were able to stratify high and low risk patients. In particular the miRNA+clinical factors model was better than the miRNA only model at stratifying patients and had a 50% improvement in specificity compared to the clinical factors alone (FIG. 3 and Table S2). This large increase in the number of patients correctly classified as low risk (true negatives) shows how a miRNA-based model could help identify patients in which adjuvant therapy could safely be withheld. With respect to other available prognostic signatures, the most current studies focus on gene expression signatures. Several RNA expression profiles are under investigation as prognostic tests for early stage (I and II) primary melanoma. DecisionDx (Castle Biosciences) is a commercially available 31-mRNA gene expression assay; however, despite being CLIA-approved in several states it is not recommended in the National Comprehensive Cancer Network Melanoma Guidelines. Recently another gene expression assay, the Merlin assay (SkylineDX) was analyzed by Amaral et al. for stage I-II patients which combines clinico-pathologic features and the expression profile (CP-GEP) of 8 genes (ITGB3, PLAT, SERPINE2, GDF15, TGFBR1, LOXL4, CXCL8 and MLANA) (Amaral et al., 2023). The CP-GEP improved stage I/IIA stratification compared to AJCC criteria alone. Notably the CP-GEP enabled identification of patients who could forgo sentinel lymph node biopsy SNLB. Yet, this study was performed on a large number of stage I patients whose data was retrospectively collected. Additionally, they did not include ulceration as a clinical factor which may be important to classify stages IIB and IIC. Similarly, in patients in stages I-III the MELAGenix score uses an 11-gene signature (KRT9, DCD, PIP, SCGB1D2, SCGB2A2, COL6A6, GBP4, KLHL41, ECRG2, HES6, MU (7) using RT-PCR to stratify high and low risk of melanoma specific death, not recurrence as our model does. The MELAGenix score is currently being assessed in a randomized prospective phase III clinical trial (NivoMela) for stage II melanoma patients stratified and treated based on their biomarker risk score. Other prognostic models are being developed using artificial intelligence (AI) machine-learning to predict patient outcome. One large study used retrospective clinical databases to classify recurrent and non-recurrent stage I-II melanomas based on patient demographics, medical history (including the Charlson comorbidity index), and tumor characteristics using an AI algorithm. Although this technique was used to classify recurrent vs non-recurrent patients, one of their key drivers in the model, the mitotic rate, is no longer reported as a clinico-pathologic factor in the latest revision to the staging system because tumor thickness and ulceration provided a more reliable outcome prediction. Another recent AI model analyzed H&E images of stage I-III to predict survival and metastasis, however this study was not performed on a prospective cohort and as indicated as one of their limitations it has yet to be tested on a larger stage II cohort. One miRNA-based test, the Melaseq signature, been proposed as a potential companion to conventional histopathological diagnosis by assessing differentially expressed miRNAs between melanoma and non-melanoma blood and solid tumor/skin samples. Other investigations have focused on the prognostic role of miRNAs. Most of these have either focused on individual miRNAs, on miRNA signatures in metastatic melanoma or on miRNA makers in circulation. But none of the above studies have focused exclusively on stage II melanoma and have combined miRNA and clinical factors, in contrast to the present disclosure.

Some differences were observed between the miRNA only model and the miRNA+clinical factors model. Most miRNA in the miRNA only model were statistically significantly associated with tumor thickness hsa-miR-200b-3p, hsa-miR-4521 and hsa-miR-93-5p. However, the miRNA+clinical factors model also had two miRNAs associated with thickness, hsa-miR-585-3p and hsa-miR-4521. hsa-miR-4521 and hsa-miR-585-3p are in the miRNA+clinical factors model, may have other roles. In other cancers, hsa-miR-585-3p has been shown to target Calpain-9 (a cysteine protease), PSME3 (encoding for a proteasome subunit) and FSCN1 (encoding for an actin-binding protein). hsa-miR-4521 had been shown to target: IGF2 and FOXM1, FAM129A.

hsa-miR-585-3p and hsa-miR-4521 may have a stronger roles in these pathways than their association with thickness and thus remain an aspect of the disclosure in the combined model along with Breslow thickness. Although hsa-miR-93-5p has been shown to signal in a multitude of pathways in other cancers, in melanoma its ability to predict recurrence is weaker than Breslow tumor thickness.

Without intending to be bound by any particular theory it is believed that this disclosure is the first to report the described miRNAs in melanoma tissues and their use with the described clinical factors.

### Materials and Methods

### Clinical Specimens

Stage II melanoma specimens (AJCC 8th edition) were obtained from the NYU Melanoma Clinicopathological-Biospecimen Database and Repository, a prospective cohort with an integrated clinicopathological-biospecimen database with over 4000 melanoma patients enrolled since 2002 (Wich et al., 2009). Clinicopathological characteristics were obtained through medical records and pathology reports and stored in a RedCap database with several layers of protection of patients' personal health information (PHI). All patients consented and had protocol-driven follow up to collect clinical data. Study approval was acquired from the Institutional Review Board at the New York University Langone Health (NYULH) (IRB #10362). Out of 198 patients, two were excluded due to the presence of multiple primaries and one sample failed NanoString analysis. In order to derive a signature using a robust signal, out of the remaining 195 samples, those with insufficient miRNA content were excluded (n=20) (details on the exclusion criteria is described in “RNA measurements using nCounter platform” below). Final analysis was performed on 175 clinical specimens (n=83 stage IIA, n=68 stage IIB and n=24 stage IIC). The samples were run across five batches based on when each specimen was obtained. Sample distribution across batch is as follows: batch 1, n=6; batch 2, n=97; batch 3, n=27; batch 4, n=26, batch 5, n=19. Additionally, four control tumor samples were run in each batch to identify batch to batch variations or changes in data distribution between runs.

### RNA Extraction

Hematoxylin and eosin (H&E) stained slides from formalin-fixed paraffin-embedded (FFPE) tumor sections (5 μm) were reviewed and annotated for histopathologic characteristics by expert dermatopathologists. The tumor area was outlined and used as a guide for macrodissecting up to 10 FFPE slides. Total RNA and DNA were extracted using the AllPrep DNA/RNA FFPE kit (Qiagen) according to manufacturer's instructions. Extracted total RNA was aliquoted and stored at −80° C. Concentrations were determined using a QuBit 2.0 Fluorometer (Invitrogen, Carlsbad, CA.) with a single-use aliquot to avoid RNA degradation of the tested sample.

RNA Measurements Using nCounter Platform

Following extraction, 100 ng (whenever possible) of total RNA were hybridized to capture and reporter probes using the NanoString nCounter® Human v3 miRNA Expression Assay. The expression assay includes 798 miRNA probes in addition to a set of internal controls (5 mRNA housekeeping gene probes, 6 positive controls, 8 negative controls, 6 ligation controls and 5 spike-in controls). The assays were run according to manufacturer's instructions on the nCounter® MAX/FLEX System and then read on the Nanostring Digital Analyzer using 280 fields of view. Quality control checks were performed using default QC parameters. As mentioned above, low quality miRNA samples were excluded from the analysis using the following cut off method. Briefly, each sample was assessed by calculating the number of miRNAs with counts greater than 50 (this count is above the highest negative control values). The average number of miRNAs with counts greater than 50 was calculated across all samples (referred to as the “mean”) along with the standard deviation (SD). The cut off for low miRNA expression in a given samples was: (mean−1.5*SD (mean)). The resulting cut off was set at 51 miRNAs. In other words, samples needed to have more than 51 miRNAs with counts greater than 50 to be included. Overall, 20 out of 195 patient samples were excluded for low miRNA expression.

### Data Pre-Processing

Imputation. There was a subset of patients for whom we analyzed both their initial biopsy sample and their excision specimen (n=17). We found differentially expressed miRNAs associated with the excision specimens that suggested their expression was related to wound healing (data not shown). Using the data of the 17 patients with both biopsy and excision samples, we fitted a linear regression model on the excision data to predict the biopsy. Then, for the 67 patients without biopsy samples, we used the fitted model to impute biopsy expression levels based on their excision samples.

Thresholding. Negative controls (n=7) were used for background thresholding. Negative control E was removed (with approval from NanoString) due to its abnormally high counts. In each of the four batches, we calculated the median count of the seven negative controls for each sample. We next computed the mean and standard deviation (SD) of the median negative control counts across the samples, and used the mean+2SD as the threshold. We separated the samples by recur/non-recur status, and computed median count of each miRNA in recur and non-recur groups, respectively. If the median count of a miRNA in a batch was equal or lower than the threshold in both groups, we excluded it from further analyses. Further analysis was done on 195 miRNAs. If any of these miRNAs were below the batch-specific threshold in a particular sample (i.e., mean+2SD), the value was replaced with the threshold value. For all downstream analyses all miRNA values and positive controls were log transformed.

Positive correction. Positive controls allow for the correction of sample-to-sample variations. In the NanoString assay, the positive controls measure the efficiency of the probe hybridization reactions. We performed batch-specific positive correction, using the following formula:

$$c \times ( \frac{m}{s} ) *$$

where c is the log (count) data of a miRNA in a given sample, m is the mean of the sums of the six positive controls across all samples in a given batch, and s is the sum of all of the six positive controls for that given sample.

Normalization. The top 40 most highly expressed miRNAs were used as a reference for normalization using the following formula:

$$c \times ( \frac{m}{s} ) **$$

where c denotes the log (count) data of miRNA, m denotes the mean of sums of the top 40 miRNAs across all samples and all batches, and s is the sum of the top 40 miRNAs for a given sample (Brumbaugh et al., 2011).

### Statistical Analysis

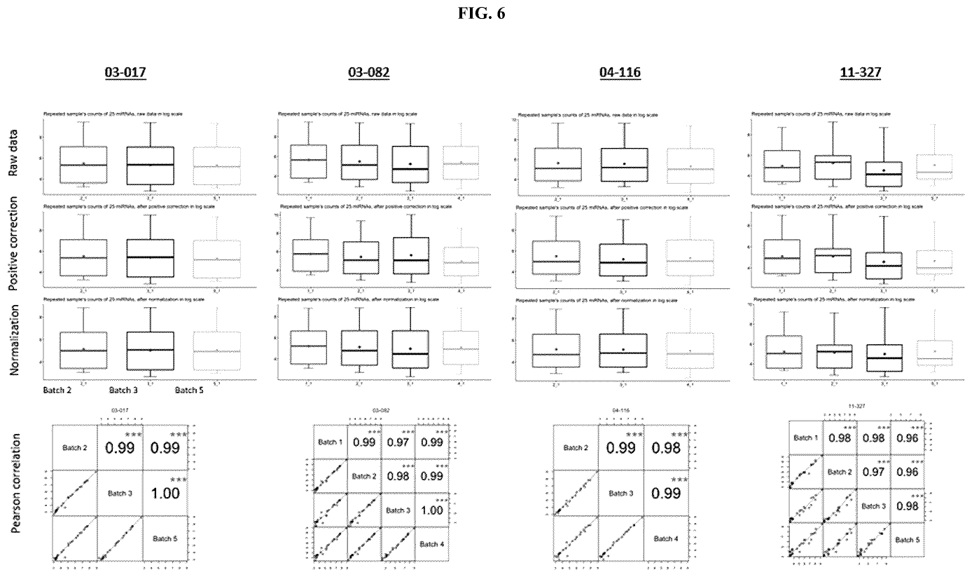

Effect consistency. To identify miRNAs with consistent effects on recurrence across all five batches, we implemented a Cox regression for each of the 195 miRNAs in each of the five batches. Due to the low number of samples in batch 1 (n=6), it was combined with batch 2 (n=97). Candidate miRNAs were selected if their coefficients were consistently observed in the same direction (positive or negative) in all batches and their p-value was less than 0.1 in at least one batch. To assess the robustness of miRNAs as sole predictors of outcome or as predictors in combination with clinical factors, two analyses were performed: one using univariate Cox regression only and another using univariate Cox regression or with adjustment for baseline covariates (Breslow thickness and ulceration). In total, 20 miRNAs were included in the study to assess them independently as prognostic factors and 25 miRNAs were used for downstream analyses in the study combining miRNA and clinical covariates as prognostic factors (Table S1). The Cox Proportional Hazard model is the following: λ(t|X₁, X₂, X₃)=λ₀(t)e^{β}^{₁}^{X}^{₁}^{+β}^{₂}^{X}^{₂}^{+β}^{₃}^{X}^{₃}, where X₁ is log thickness, X₂ is ulceration, X₃ is each miRNA from the 195 miRNAs. As a quality check, we examined the consistency and reliability of the counts of the selected miRNAs among the four control samples across all batches (FIG. 6). Due to technical reasons, counts in batch 5 were slightly higher than the other batches. Batch 5 (n=19 patients) was then removed from model building and only used for testing.

Training and test sets. To ensure robust model building, samples from batches 1-4 were split into a training set and a separate validation test set to measure the prediction accuracy of the built model. Samples were assigned into each set using block randomization to ensure the ratios of recurred vs non-recurred patients were similar in both the training and test sets. Briefly, we used logistic modelling with recurrence as the outcome, and age at diagnosis, sex, log tumor thickness, and ulceration as predictors to create propensity scores. The propensity score was applied to balance the patients' clinical characteristics according to their recurrence status (recurred/did not recur) in the training and test sets. Subjects were separated into recurrence and non-recurrence groups and ranked based on their propensity scores from high-to-low in each group. Then, three patients were placed into a set according to the ranks (e.g., in the recurrence group, rank 1, 2, 3 as a set and 4, 5, 6 as a set, etc.). Finally, two patients from each set were randomly selected in the recurrence and non-recurrence groups to create the training set. The remaining patients comprised the testing set. In the end, 103/155 (⅔ of batch 1-4) samples were assigned to the training set. The set-aside test set comprised 52/155 (⅓ of batch 1-4) samples+19 samples from batch 5, for a total of 71 samples.

Identification of candidate miRNAs. We used Cox models to screen for miRNAs significantly (p<0.05) associated with time to recurrence in the training set. To identify miRNA candidates independent of clinical factors, the analysis was performed on the 20 miRNAs previously chosen for their consistent effect across all five batches (without clinical factors). We fit Cox models for each of the 20 miRNAs without adjustment for clinical factors. To identify miRNA candidates for analysis with clinical covariates the analysis was performed on the previously chosen 25 miRNAs which had consistent effects in all 5 batches with or without clinical factors. We fit Cox models for each of the 25 miRNAs either: i) without adjustment for clinical factors (age at diagnosis, sex, log thickness, and ulceration) or ii) with adjustment for clinical factors. To further reduce the dimension of miRNAs, we applied Cox regression with LASSO variable selection using 3-fold cross validation for tuning parameters. Since the tuning parameters are based on cross validation, the selected miRNAs can depend on how the dataset is partitioned. So, we repeated the LASSO variable selection 50 times, and kept the miRNAs which were frequently selected (>30 times). Log Breslow thickness and ulceration were included for adjustment only in the combined miRNA+clinical factors model which used the pre-selected 25 miRNAs. The Cox LASSO model: λ(t|X)=λ₀(t)e^{Σ}^{ⱼ}^{β}^{ⱼ}^{X}^{ⱼ}, where X=(x₁, . . . ,xₚ) are the clinical factor and 25 candidate miRNAs, λ₀(t) is the baseline hazard function, β=(β₁, . . . ,βₚ) are the parameters need to be estimated using the standard LASSO method. The partial likelihood for Cox model is:

$$L ( β ) = {\prod }_{r ∈ D} \frac{exp ( {β}^{T} {x}_{{j}_{r}} )}{\{ {\sum }_{j ∈ {R}_{r}} exp ( {β}^{T} {x}_{{j}_{r}} ) \}} ,$$

where D is the set of indices of the events, Rᵣ is the set of indices of the at risk set at time tᵣ. The parameters β are estimated using standard maximization method via the criterion \hat{β}=argmin log L(β), subject to Σ|βⱼ|≤s, where s>0 is a user-specified parameter.

Identify a consistent miRNA signature. Because signature development depends on the composition of the training and test sets, we repeated the sampling strategy described above to generate 50 sub-training and 50 sub-testing sets. For each iteration, the training set was used to identify a miRNA signature for predicting survival status, and the test set was used to assess its performance. We calculated the Area Under the Receiver Operating Characteristic Curve (AUC) on each of the training and test sets. This resulted in 50 miRNA signatures. The miRNAs present in 10 or more signatures were used for further refinement (Table S3).

Refining the miRNA recurrence prediction model with and without clinical covariates. The training set (n=103) was split again into sub-training (80%) and sub-testing sets (20%) using block randomization. This process was repeated 20 times to generate 20 sub-training and 20 sub-testing groups. Note that none of the patients in the set-aside test set (defined above, n=71) were used to create sub-training or sub-testing groups. For each sub-training set we developed a Cox model with LASSO, using the miRNAs selected using the procedures described above, to predict time to recurrence and test its performance using the sub-testing set. For each model we derived a receiver operating characteristic (ROC) curve and computed their corresponding area under curves (AUCs) for landmark recurrence time points (i.e. 24, 36, 48 and 60 months). The means, medians, standard deviations (SDs), and ranges of the AUCs were calculated for each model. Then we ranked the models using the mean and median AUCs from highest to lowest, and using SD and range from least to largest. Then, we calculated the geometric mean (geomeans) of those four ranks for each time point of each model. A model with a smaller geomean of ranks was considered the most stable model (e.g., geomean=1 means the model's mean, median, SD and range of test AUCs were all ranked 1st). The models (miRNA only; miRNA+clinical factors) with the smallest geomean of ranks among the 20 candidate models were selected as the final models, and were chosen for evaluation in the set-aside test set. The miRNAs and clinical covariates included in these models and shown in Table 2. To evaluate the classification accuracy of the recurrence prediction models at 3 years (36 months), the cutoff for high vs. low risk was set using sensitivity to detect recurrence of 0.9 in order to generate Kaplan-Meier and Waterfall plots for each of the models (FIGS. 2, and 3).

The following equations are included in this disclosure.

To narrow the miRNAs for further study, the following formula was applied to a set of 195 miRNAs that passed the pre-processing step. This yield 25 miRNAs for further study.
The Cox model:
   ΔtX1,X2,X3=λ0teβ1X1+β2X2+β3X3,
   Where X1 is log thickness, X2 is ulceration, X3 is each miRNA from the 195 miRNAs.

To further reduce the dimension of miRNAs, we applied Cox regression with LASSO variable selection method using cross validation for tuning parameters.
The Cox LASSO model* **:
   λtX=λ0tejβjXj, where X=(x1, . . . ,xp) are the clinical factor and 25 candidate miRNAs, Δ0t is the baseline hazard function, β=(β1, . . . ,βp) are the parameters need to be estimated.
   The partial likelihood: Lβ=r∈Dexp(βTxjr){j∈Rrexp(βTxj)}, where D is the set of indices of the events, Rᵣ is the set of indices of the at risk set at time tᵣ.
   The parameters β are estimated via the criterion β=argminlog Lβ, subject to |βj|≤s, where s>0 is a user-specified parameter.
*Tibshirani R. The lasso method for variable selection in the Cox model. Statistics in medicine. 1997 Feb. 28; 16 (4): 385-95.
** Hastie T, Qian J. Glmnet vignette. Retrieved June. 2014 June; 9 (2016): 1-30.

To derive the coefficients for the risk score model we did the following, based on the same references mentioned above:
λtX=λ0tejβjXj, where X=(x1, . . . ,xp) are the clinical factor and 10 candidate miRNAs, λ0t is the baseline hazard function, β=(β1, . . . ,βp) are the parameters need to be estimated.
The partial likelihood: Lβ=r∈Dexp(βTxjr){j∈Rrexp(βTx)}, where D is the set of indices of the events, Rᵣ is the set of indices of the at risk set at time tᵣ.
The parameters β are estimated via the criterion β=argminlog Lβ, subject to |βj|≤s, where s>0 is a user-specified parameter.

The risk score is calculated using:

−0.389*hsa.miR.585.3p−1.68*hsa.miR.4521+0.555*hsa.miR.575+0.573*log(thickness)+0.764*ulceration

TABLE 1
Summary of clinicopathological characteristics of melanoma tissues from Stage II patients

| | Overall | | Recurred | | Did not recur | | p-value | Missing (%) |
|---|---|---|---|---|---|---|---|---|
| n | 175 | | 52 | | 123 | | | |
| Age at Initial Diagnosis | 70.0 | [57.5, 77.0] | 65.0 | [53.5, 75.2] | 71.0 | [59.5, 78.0] | 0.135 | 0 |
| (median [IQR]) | | | | | | | | |

Sex (%)

| | | | | | | | | |
|---|---|---|---|---|---|---|---|---|
| F | 78 | (44.6) | 24 | (46.2) | 54 | (43.9) | 0.914 | 0 |
| M | 97 | (55.4) | 28 | (53.8) | 69 | (56.1) | | |

Race (%)

| | | | | | | | | |
|---|---|---|---|---|---|---|---|---|
| White | 166 | (96.5) | 48 | (92.3) | 118 | (98.3) | 0.195 | 1.7 |
| Black | 1 | (0.6) | 1 | (1.9) | 0 | (0.0) | | |
| Hispanic | 2 | (1.2) | 1 | (1.9) | 1 | (0.8) | | |
| Other | 3 | (1.7) | 2 | (3.8) | 1 | (0.8) | | |

Initial Stage (%)

| | | | | | | | | |
|---|---|---|---|---|---|---|---|---|
| IIA | 83 | (47.4) | 16 | (30.8) | 67 | (54.5) | 0.011 | 0 |
| IIB | 68 | (38.9) | 25 | (48.1) | 43 | (35.0) | | |
| IIC | 24 | (13.7) | 11 | (21.2) | 13 | (10.6) | | |
| Thickness (mm) (median | 2.7 | [2.1, 4.0] | 3.5 | [2.3, 4.3] | 2.5 | [2.0, 3.8] | 0.003 | 0 |
| [IQR]) | | | | | | | | |

Ulceration (%)

| | | | | | | | | |
|---|---|---|---|---|---|---|---|---|
| Yes | 106 | (60.6) | 34 | (65.4) | 72 | (58.5) | 0.498 | 0 |
| No | 69 | (39.4) | 18 | (34.6) | 51 | (41.5) | | |

TILS (%)

| | | | | | | | | |
|---|---|---|---|---|---|---|---|---|
| A | 57 | (35.2) | 21 | (42.0) | 36 | (32.1) | 0.495 | 7.4 |
| B | 21 | (13.0) | 4 | (8.0) | 17 | (15.2) | | |
| NB | 81 | (50.0) | 24 | (48.0) | 57 | (50.9) | | |
| unknown | 3 | (1.9) | 1 | (2.0) | 2 | (1.8) | | |
| Mitotic Index (median | 5.0 | [2.0, 8.0] | 6.0 | [3.0, 8.8] | 4.0 | [2.0, 8.0] | 0.066 | 2.9 |
| [IQR]) | | | | | | | | |

Mitotic Index (%)

| | | | | | | | | |
|---|---|---|---|---|---|---|---|---|
| <5/mm\hat{ }2 | 83 | (48.8) | 19 | (38.0) | 64 | (53.3) | 0.098 | 2.9 |
| >=5/mm\hat{ }2 | 87 | (51.2) | 31 | (62.0) | 56 | (46.7) | | |
| Time to first recurrence | 18.0 | [13.0, 30.0] | 18.0 | [13.0, 30.0] | NA | [NA, NA] | NA | 71.4 |
| (months) (median [IQR]) | | | | | | | | |

Status (%)

| | | | | | | | | |
|---|---|---|---|---|---|---|---|---|
| Alive | 138 | (78.9) | 32 | (61.5) | 106 | (86.2) | 0.001 | 0 |
| Died | 37 | (21.1) | 20 | (38.5) | 17 | (13.8) | | |
| Time from initial | 64.0 | [39.5, 89.5] | 66.5 | [38.5, 85.5] | 61.0 | [39.5, 89.5] | 0.961 | 0 |
| diagnosis to last follow up | | | | | | | | |
| (months) (median [IQR]) | | | | | | | | |

TABLE 2
Final miRNAs and clinical factors included
in the recurrence-prediction model

| miRNA only model | | miRNA and clinical factors model | |
|---|---|---|---|
| | Coefficient | | Coefficient |
| hsa-miR-200b-3p | 0.2484 | hsa-miR-585-3p | −0.389 |
| hsa-miR-4521 | −0.4193 | hsa-miR-4521 | −1.68 |
| hsa-miR-575 | 0.4267 | hsa-miR-575 | 0.555 |
| hsa-miR-93-5p | 0.3908 | Breslow thickness (log) | 0.573 |
| hsa-miR-29c-3p | −0.1216 | Ulceration (Present) | 0.764 |

TABLE S1
Candidate miRNAs derived with and without clinical factors. Candidate
miRNAs out of 195 selected if their coefficients were consistently
observed in the same direction (positive or negative) in all batches
and their p-value was less than 0.1 in at least one of the batches
(See Effect Consistency in Materials and Methods). Two analyses
were performed: one using univariate Cox regression only and another
using univariate Cox regression or after adjusting for baseline
covariates (Breslow thickness and ulceration).

| Candidate miRNA derived without | Candidate miRNA derived with |
|---|---|
| clinical covariates (n = 20) | clinical covariates (n = 25) |
| hsa-let-7g-5p | hsa-let-7g-5p |
| hsa-let-7i-5p | hsa-let-7i-5p |
| hsa-miR.223.3p | hsa-miR.223.3p |
| hsa-miR-25-3p | hsa-miR-25-3p |
| hsa-miR-148a-3p | hsa-miR-148a-3p |
| hsa-miR-93-5p | hsa-miR-93-5p |
| hsa-miR-106b-5p | hsa-miR-106b-5p |
| hsa-miR-32-5p | hsa-miR-32-5p |
| hsa-miR-29c-3p | hsa-miR-29c-3p |
| hsa-miR-185-5p | hsa-miR-185-5p |
| hsa-miR-575 | hsa-miR-575 |
| hsa-miR-378e | hsa-miR-378e |
| hsa-miR-200b-3p | hsa-miR-200b-3p |
| hsa-miR-4521 | hsa-miR-4521 |
| hsa-miR-551a | hsa-miR-551a |
| hsa-miR-27a-3p | hsa-miR-27a-3p |
| hsa-miR-448 | hsa-miR-448 |
| hsa-miR-337-3p | hsa-miR-337-3p |
| hsa-miR-127-3p | hsa-miR-127-3p |
| hsa-miR-585-3p | hsa-miR-585-3p |
| | hsa-let-7a-5p |
| | hsa-miR-126-3p |
| | hsa-miR-150-5p |
| | hsa-miR-1234-3p |
| | hsa-miR-4521 |

TABLE S2
Specificity and sensitivity of the test set values derived
from the 3-year (36-month) recurrence prediction clinical
and miRNA + clinical models using MinValueSe at 0.90.

| | | | Clinical factors + |
|---|---|---|---|
| | Clinical factors only | miRNA only | miRNAs |
| | Estimate (%) | Estimate (%) | Estimate (%) |
| Sensitivity | 94.6 | 90.0 | 90.2 |
| Specificity | 5.7 | 46.2 | 55.9 |
| PPV | 25.4 | 35.5 | 40.0 |
| NPV | 75.0 | 92.3 | 93.5 |

PPV = Positive Predictive Value; NPV = Negative Predictive Value.

TABLE S3
List of miRNA found in more than 10 miRNA signatures.
Frequency indicates the number of signatures (out
of 50) in which the particular miRNA was included.

| | |
|---|---|
| miRNA only | miRNA + Clinical factors |

| miRNA | Frequency | miRNA | Frequency |
|---|---|---|---|
| hsa.miR.93.5p | 45 | hsa.miR.1234.3p | 48 |
| hsa.miR.575 | 27 | hsa.miR.29c.3p | 44 |
| hsa.miR.29c.3p | 20 | hsa.miR.585.3p | 36 |
| hsa.let.7g.5p | 15 | hsa.miR.93.5p | 32 |
| hsa.miR.200b.3p | 13 | hsa.miR.4521 | 23 |
| hsa.miR.4521 | 12 | hsa.let.7g.5p | 20 |
| hsa.miR.585.3p | 11 | hsa.miR.1180.3p | 18 |
| | | hsa.miR.575 | 12 |
| | | hsa.let.7a.5p | 11 |
| | | hsa.miR.27a.3p | 11 |

The disclosure includes the following aspects, which are not intended to be limiting.

The described recurrence prediction model is based on the measurement of miRNA counts in total RNA extracted from formalin-fixed, paraffin-embedded primary melanoma tumors from patients with AJCC stage II melanoma. The process begins with microscopic review of a hematoxylin and eosin-stained slide to identify the tumor and plan a macrodissection procedure in which surrounding normal tissue will be removed from the tumor cells on the slide. Approximately 10 unstained sections of tumor material undergo macrodissection, and the remaining tumor material is scraped into a tube. The tumor material undergoes standard processing to extract nucleic acids using the AllPrep DNA/RNA FFPE kit (Qiagen) according to manufacturer's instructions. RNA concentrations are determined and a minimum of 90 ng RNA are hybridized to capture and reporter probes using the Nanostring nCounter® Human v3 miRNA Expression Assay according to manufacturer's instructions.

Raw data for each sample (i.e. miRNA counts of ˜798 miRNAs plus controls) are manually reviewed for quality measures, and samples meeting the quality metrics are then analyzed. The analytical method includes thresholding, log transformation of miRNA counts, positive correction, and normalization using the top 40 expressed genes.

Using a statistical method we identified miRNAs that were differentially expressed in patients who suffered a melanoma recurrence compared to those who did not recur. Patients who did not recur were followed for a minimum of 3 years. The statistical characteristics of these miRNAs were combined with AJCC staging criteria (i.e. tumor thickness and ulceration) into a multivariable model to predict melanoma recurrence. The model contained 3 miRNAs plus log tumor thickness and ulceration. It substantially outperformed the model that used only the staging criteria.

In December 2021, adjuvant pembrolizumab (an anti PD-1 immunotherapy) was approved for the treatment of patients with completely resected stages IIB and IIC melanoma. The approval was based on the KEYNOTE-716 randomized clinical trial (n=976), which demonstrated an improvement in 18-month recurrence-free survival for pembrolizumab (85.8%) compared with placebo (77.0%). Close examination of these findings reveals that the number needed-to-treat (NNT) to prevent one recurrence was 11:1. The main factor driving this ratio is not the lack of effectiveness of the treatment, but the relatively low recurrence rate in the placebo group (23%). Given the treatment-related toxicity risks (16% grade 3-4 events that led to treatment discontinuation in the trial) and the very high costs of immunotherapy, the disclosure addresses a need to more accurately identify stage II melanoma patients who may benefit from adjuvant therapy, and distinguish them from surgically cured patients who do not need it. In December 2023, adjuvant nivolumab (another anti PD-1 immunotherapy) was also approved by the FDA for the treatment of completely resected stage IIB and stage IIC melanoma. The approval was based on the CheckMate 76K trial that demonstrated very similar results to the KEYNOTE-716 trial. Unfortunately, the commercial molecular tests that have been developed to guide the management of localized, completely resected stage I and stage II melanoma are unproven, as evidenced by the lack of recommendation by the National Comprehensive Cancer Network Melanoma Guidelines, and the American Academy of Dermatology Melanoma Guidelines. Taken together, these factors substantially elevate the importance of developing new prognostic biomarkers for stage II melanoma, such as that of the present disclosure.

The current disclosure includes the following improvements over previous approaches, which are not meant to be limiting: The disclosure measures miRNA, which is more stable in FFPE material than mRNA, so less subject to sample-to-sample variations in degradation. It includes a new statistical model developed only stage II melanoma patients, which is the intended-use population for the model. The patient accrual method was prospective, not a case-control study. This means the characteristics of the patients closely match the general melanoma population, and performance characteristics of the model are likely to be reproduced in another cohort of prospectively accrued melanoma patients. In contrast, there are commercial mRNA-based assays to predict recurrence in early stage melanoma, but they were developed using a mix of stage I, II and III melanoma, not exclusively stage II melanoma. They have not convincingly demonstrated that they improve the prediction of melanoma recurrence in stage II patients better than a clinical model alone. For example, the commercially available CASTLE DECISIONDX-MELANOMA model seeks to predict melanoma recurrence; the commercially available Merlin assay (SKYLINE DX) seeks to predict the likelihood of melanoma spread to regional lymph nodes (i.e. sentinel lymph node) at the time of initial staging. The Merlin assay is focused on a completely different indication than the presently described assay. Patients who studied in this disclosure had all undergone standard staging work up including investigation of their sentinel lymph nodes, and were all negative.

While the present disclosure has some similarities to a study with the InterMEL group. that study design was quite different from the present disclosure. The InterMEL study used a case-control design, and samples were collected retrospectively based on availability of tumor and clinical information at each participating center. In addition, their endpoint was death within 5 years of diagnosis (cases) versus alive at 5 years (controls). This design is less clinically relevant than the present disclosure because the current clinical need is to more accurately identify patients at high (or low) risk of recurrence when deciding to administer adjuvant therapy, which the present disclosure addresses.

What is claimed is:

1. A method comprising testing a sample of a patient's Stage II cutaneous melanoma tumor to determine if the patient is at risk for relapse of the melanoma, the testing comprising determining a change in microRNAs in the sample relative to a value for the same microRNAs obtained from individuals who did not experience relapse of the Stage II cutaneous melanoma, wherein the microRNAs comprise:
i) a combination of microRNAs selected from the group consisting of hsa.miR.93.5p, hsa.miR.575, hsa.miR.29c.3p, hsa.let.7g.5p, hsa.miR.200b.3p, hsa.miR.4521, and hsa.miR.585.3p; or
ii) a combination of microRNAs selected from the group consisting of hsa.miR.1234.3p, hsa.miR.29c.3p, hsa.miR.585.3p, hsa.miR.93.5p, hsa.miR.4521, hsa.let.7g.5p, hsa.miR.1180.3p, hsa.miR.575, hsa.let.7a.5p, and hsa.miR.27a.3p.

2. The method of claim 1, wherein the microRNAs include the combination of ii), the method further comprising determining clinical factors of the tumor that include Breslow thickness and ulceration.

3. The method of claim 2, comprising determining a change in the microRNAs and a change in the clinical factors relative to a value for the microRNAs and the clinical factors obtained from individuals who did not experience relapse of the Stage II cutaneous melanoma to determine that the individual is at risk of melanoma relapse, and wherein the change in the microRNAs and a change in the clinical factors determined from the patient provides a relapse risk score.

4. The method of claim 3, further comprising administering to the individual an immune checkpoint therapy.

5. The method of claim 4, wherein the immune checkpoint therapy comprises anti-PD-1 immunotherapy.

6. A method comprising administering immune checkpoint therapy to an individual who has been determined to have a risk of recurrence of Stage II cutaneous melanoma, wherein said risk is determined using the method of claim 1.

7. The method of claim 6, wherein the microRNAs include the combination of ii) of claim 1, the method further comprising determining clinical factors of the tumor that include Breslow thickness and ulceration.

8. The method of claim 7, comprising determining a change in the microRNAs and a change in the clinical factors relative to a value for the microRNAs and the clinical factors obtained from individuals who did not experience relapse of the Stage II cutaneous melanoma to determine that the individual is at risk of melanoma relapse, and wherein the change in the microRNAs and a change in the clinical factors determined from the patient provides a relapse risk score.

9. The method of claim 8, wherein the immune checkpoint therapy comprises anti-PD-1 immunotherapy.
